(19) United States
(12) Patent Application Publication
(10) Pub. No.: US 2008/0139500 A1
(43) Pub. Date: Jun. 12, 2008

(54) Use of composite monomer or monomers, catalyst, sodium hyaluronate or hyaluronic acid, glucosamide sulfate, chondroiten sulfate or chlorides, for the treatment of osteoarthritis by intraarticular application to a hip or knee joint

(76) Inventor: Isadore Elliott Goldberg, Coconut Creek, FL (US)

Correspondence Address: ISADORE ELLIOTT GOLDBERG, Apt F-1, 2402 Antigua Circle, Coconut Creek, FL, 33066, US

(21) Appl. No.: 11/636,689

(22) Filed: Dec. 11, 2006

(51) Int. Cl. A61K 31/715; A61P 19/02

(52) U.S. Cl. 514/54

(57) ABSTRACT

This invention relates to the methods of treatment of osteoarthritis of the hip or knee joint by intraarticular application of a mixture of certain monomer or monomers, catalyst, sodium hyaluronate, glucosamine sulfate, and chondroiten sulfate.

Use of composite monomer or monomers, catalyst, sodium hyaluronate or hyaluronic acid, glucosamide sulfate, chondroiten sulfate or chlorides, for the treatment of osteoarthritis by intraarticular application to a hip or knee joint.

### REFERENCES CITED (REFERENCED BY)

| | | |
|---|---|---|
| 4217260 | August 1980 | Daniel et al. |
| 4636524 | January 1987 | Balazs et al. |
| 4698360 | October 1987 | Masquelier |
| 4713448 | December 1987 | Balazs et al. |
| 4808576 | February 1989 | Shults et al. |
| 4801619 | January 1989 | Lindblad |
| 5099013 | March 1992 | Balazs et al. |
| 5409904 | April 1995 | Hecht et al. |
| 5633003 | May 1997 | Cantor |
| 6037331 | March 2000 | Shalaby et al. |
| 6645256 | November 2003 | Saduja et al. |
| 6756419 | June 2004 | Sanduja et al. |
| 6497786 | December 2002 | Kilgore et al. |

### FIELD OF INVENTION

This Invention is related to methods of composition for the treatment of osteoarthrittis of the hip and knee joint by means of the intraarticular application of a mixture of monomer or monomers such as acrylics, methacrylics, polyester, epoxie, amines, styrene, etc.; and sodium hyaluronate or hyaluronic acid, gluconate sulfate, chondroiten sulfate or the corresponding chlorides. The material is injected, allowed to polymerize while moving the femur, and when fully polymerized, is designed to withstand and resist a compressive articulating load placed on the joint.

In order to avoid hip joint or knee joint replacement surgery, my system provides, after intraarticulate injection of the above described mixture, while moving the femur. When fully polymerized, will form a low friction, low wear, lubricated, composite material that will not adhere to the hip socket or femur, will stay in place when the femur is rotated in the socket, is flexible, prosthetic bearing, durable, strong, long lasting, non toxic and bio compatible to humans. The use of materials, such as sodium hyaluronate or hyaluronic acid, gluconate sulfate and cchondroiten sulfate or chlorides without the monomer has resulted in a poor quality tissue that can not support the weighted load. This system, after injection and when fully polymerized, produces a compatible substitute of the articular cartilage of the hip and knee, and gives excellent results and a marked reduction of pain or no pain when the femur is rotated in the socket.

A longer term advantage of this system is that it is an inductor of chondrogenesis and allows regeneration of the cartilage destroyed by osteoarthritis.

### BACKGROUND OF THE INVENTION

Osteoarthritis of the hip or knee is a condition that affects many millions of persons throughout the world. The usual treatment to relieve the pain is surgery. Such surgery is expensive, there is an extended recuperation period and not everyone is eligible or able to cope with such surgery and the possibility of the titanium ball moving from the socket. The disease of the hips or knees occurs with the gradual degeneration and destruction of the cartilage. The clinical manifestations of the development of this condition are: pain, functional disability that gradually and steadily hinders mobility; the process of walking becomes painful. As the condition worsens. It becomes more painful to walk even a short distance.

### SUMMARY OF THE INVENTION

This invention was developed to solve the problem related to the treatment of osteoarthritis of the hip or knee joint. This invention introduces a system to avoid hip or knee joint replacement surgery whereby a sterile mixture is introduced through the intraarticular implantation of monomer or monomers, sodium hyaluronate, glucosamine sulfate and chondroiten sulfate to the hip or knee joint. The monomers polymerize in place and form the equivalent of normal cartilage which serves to lubricate the hip or knee joint with a marked reduction of pain or no pain, is non toxic to humans and permits the regeneration of natural cartilage.

1. A therapeutic method for the treatment of a hip or knee joint exhibiting degeneration of the joint cartilage by the intraarticular administration to the hip or knee joint of a composition of a sterile, non-pyrogenic mixturer or monomers, catalyst, sodium hyaluronate or hyaluronic acid, chondroiten sulfate, glucosamine sulfate or hydrochlorides and causing the monomer or monomers to polymerize.

2. The method of claim 1, wherein the hip or knee joint degeneration of the cartilage is caused by osteoarthritis or some similar degeneration.

3. The method of claim no. 1 or 2 wherein the monomer or monomers may consist of acrylics, methacrylates, polyester, epoxies, amines, urethanes, styrenes.alkyds, silicones, etc. The composition of the monomer or monomers may be at a concentration of 1% to 95% by weight. The properties of the material deposited depend upon the combination of monomers, the quantity of catalyst used, etc.

4. The use of monomer or monomers used in the hip or knee joint, after polymerization, provides a strong, flexible, prosthetic bearing material that will support the weighted load, with the normal movement of the hip or knee.

5. Acrylic resins may be crosslinked with polyesters, epoxies, amines, urethanes, etc.; to improve its characteristics and obtain hardness, toughness, mar resistance, flexibility, resistance to local environment and good performance qualities.

6. The degree of protection expected is a minimum of five years and the shelf life of the material is a minimum of six months.

7. The method of claim 1, wherein the sodium hyaluronate is at a concentration of 1% to 25% and the concentration of glucosamine sulfate is at a concentration of 1% to 25% by weight and the chondroiten sulfate is at a concentration of 1% to 25%; the sodium hyaluronate may be substituted for hyaluronic acid, the glucosamine sulfate and chondroiten sulfate may be substituted for by the corresponding chlorides.

8. The method of claim Nos., 1, 2, 3, 4, 5, 6, and 7 permit the monomers to be varied considerably to permit the required amount of hyaluronait, glucosamine and chondroiten to be included in the mix.

9. The method of claim Nos. 1, 2, 7 and 8 wherein the viscosity may vary as follows:
glucosamine and chondroiten sulfate—18,000 to 55,000 daltons;
sodium hyaluronate—400,000 to 1,100.000 daltons.

10. The method of claim nos. 1, 2, 3, 4, 5, 6, 7, 8 and 9 wherein from) 0.5 to 5.0 cubic centimeters of the sterile composition is injected into the affected joint.

11. The method of claim No. 10, wherein 2 to 5 doses of the sterile composition can be administered over a three month period into the affected hip or knee joint.
